(19) United States
(12) Patent Application Publication
(10) Pub. No.: US 2008/0073208 A1
(43) Pub. Date: Mar. 27, 2008

(54) Biosensor Requiring No Code Card

(76) Inventors: Chia-Nan Wang, Hsinchu City (TW); Chia-Te Lai, Hsinchu City (TW)

Correspondence Address: WPAT, PC, 7225 BEVERLY ST., ANNANDALE, VA, 22003, US

(21) Appl. No.: 11/530,206

(22) Filed: Sep. 8, 2006

(51) Int. Cl. G01N 27/26; G01N 33/487

(52) U.S. Cl. 204/406; 204/403.01

(57) ABSTRACT

The present invention relates to a biosensor requiring no code card, which can be substantially a glucose monitor having a top key, a bottom key and a confirmation key arranged thereon for using the aforesaid top and bottom keys to control and select a set of English alphabet or numeral in a circulating manner and thereafter confirming and inputting a specific series of alphabets or numerals by pressing the confirmation key, whereas the specific series of alphabets or numerals is corresponding to a set of parameters stored in a parameter referencing unit arranged inside the glucose monitor and can be find on the package of a test strip. As soon as the specific series of alphabets or numerals is inputted into the glucose monitor, the set of parameter corresponding thereto is send to a micro processing unit for using the same to perform a setting and calibration operation so as to enhancing the accuracy of the glucose monitor. In a preferred embodiment, the top and the bottom keys can be replaced and substituted by a left key and a right key.

### FIELD OF THE INVENTION

The present invention relates to a biosensor which is operable without the use of code cards, and more particularly, to a biosensor that can be configured without the used of any test card and thus can be operated with less consumptive material requirement.

### BACKGROUND OF THE INVENTION

Following the advance of medical technology, most diseases can be cured or prevented enabling people, male or female, have a longer life than ever. Nowadays, people can do self-inspection by means of some scientific inspection methods and provide data of inspection for doctors to treat the diseases properly.

Diabetes is one of the modern diseases, which is caused by the pancreas unable to secrete insulin (or insufficient insulin). Insulin decomposes glucose in human's blood and transfers it into useful energy. The diabetes patient cannot effectively decompose glucose such that the glucose is accumulated in blood increasing the probability of apoplexy, myocardial infarction, blindness and kidney exhaustion. Therefore, the diabetes patient has to take medicine regularly and go to hospital for follow-up treatment to prevent related complications.

Laboratory tests, such as blood test and urine test, are tools helpful in evaluating the health status of an individual. Any unusual or abnormal results should be discussed with your physician. It is not possible to diagnose or treat any disease or problem with this blood test alone. It can, however, help you to learn more about your body and detect potential problems in early stages.

In order to track the Glycemic Index (GI) of any diabetes patient at any time for controlling the progress of the decease accurately, most patients adopt a kind of portable handheld glucose monitor (biosensor) for detecting and recording his/her Glycemic Index. It is noted that any of the aforesaid portable handheld glucose monitor currently available is not operable without a matching code card, since the parameters corresponding to the characteristics of test strips are stored in the code card so that the handheld glucose monitor can be configured to matching the test strips with respect to its manufacturing date and thus is calibrated, whereas the test strip holding an enzyme therein is used for receiving blood of a patient to be test by the glucose monitor. Thus, each and every test strip will required a code card specifically manufactured corresponding thereto that will cause the manufacture cost and time to increase and thus is considered to be an advantage requiring to be overcome.

### SUMMARY OF THE INVENTION

In view of the disadvantages of prior art, the primary object of the present invention is to provide a biosensor requiring no code card, which can be substantially a glucose monitor having a top key, a bottom key and a confirmation key arranged thereon for using the aforesaid top and bottom keys to control and select a set of English alphabet or numeral in a circulating manner and thereafter confirming and inputting a specific series of alphabets or numerals by pressing the confirmation key, whereas the specific series of alphabets or numerals is corresponding to a set of parameters stored in a parameter referencing unit arranged inside the glucose monitor and can be find on the package of a test strip. As soon as the specific series of alphabets or numerals is inputted into the glucose monitor, the set of parameter corresponding thereto is send to a micro processing unit for using the same to perform a setting and calibration operation so as to enhancing the accuracy of the glucose monitor.

It is another object of the invention is to provide a glucose monitor having a plurality of keys arranged thereon for using the same to input a specific series of English alphabets or numerals by pressing, whereas the specific series of alphabets or numerals is corresponding to a set of parameters stored in a parameter referencing unit arranged inside the glucose monitor and can be find on the package of a test strip. As soon as the specific series of alphabets or numerals is inputted into the glucose monitor, the set of parameter corresponding thereto is send to a micro processing unit for using the same to perform a setting and calibration operation so as to enhancing the accuracy of the glucose monitor.

Yet, another object of the invention is to provide a biosensor capable of achieving multiple analyte detections in physiological fluids, e.g. blood test or urine test, for measuring glucose, urea, cholesterol, calcium, pyruvate, serum bilirubin, and so on.

To achieve the above objects, the present invention provides a biosensor requiring no code card, comprising: a micro processing unit; a memory unit; a display unit; a power unit; a test strip; a test strip interface, for receiving the test strip; a plurality of keys, for inputting a specific series of characters; and a parameter referencing unit, for receiving and matching the inputted specific series of characters to a parameter stored therein while sending the matched parameter to the micro processing unit for enabling the same to perform a calibration operation upon the biosensor.

Other aspects and advantages of the present invention will become apparent from the following detailed description, taken in conjunction with the accompanying drawings, illustrating by way of example the principles of the present invention.

### BRIEF DESCRIPTION OF THE DRAWINGS

FIG. 1 is a schematic view of a biosensor requiring no code card according to a first embodiment of the invention.

FIG. 2 is a schematic view of a biosensor requiring no code card according to a second embodiment of the invention.

FIG. 3 is a schematic view of a biosensor requiring no code card according to a third embodiment of the invention.

FIG. 4 is a schematic view of a biosensor requiring no code card according to a fourth embodiment of the invention.

FIG. 5 is a first function block diagram of a biosensor requiring no code card according to the present invention.

FIG. 6 is a second function block diagram of a biosensor requiring no code card according to the present invention.

FIG. 7 is a reference table stored in a parameter referencing unit of a biosensor according to the present invention.

### DESCRIPTION OF THE PREFERRED EMBODIMENT

For your esteemed members of reviewing committee to further understand and recognize the fulfilled functions and structural characteristics of the invention, several preferable embodiments cooperating with detailed description are presented as the follows.

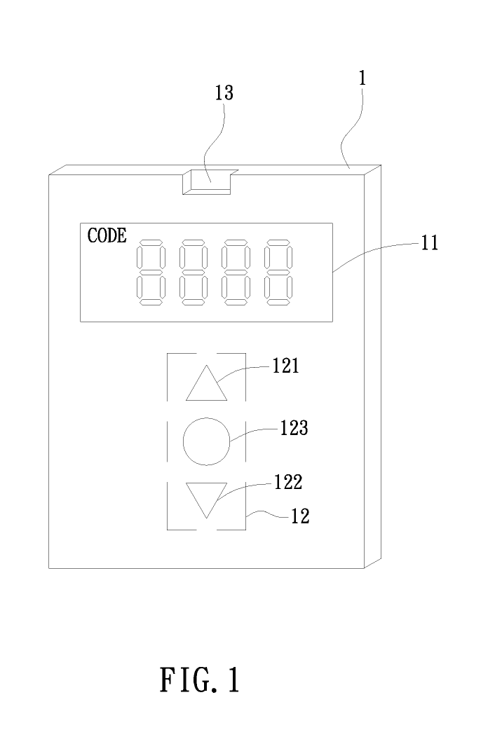

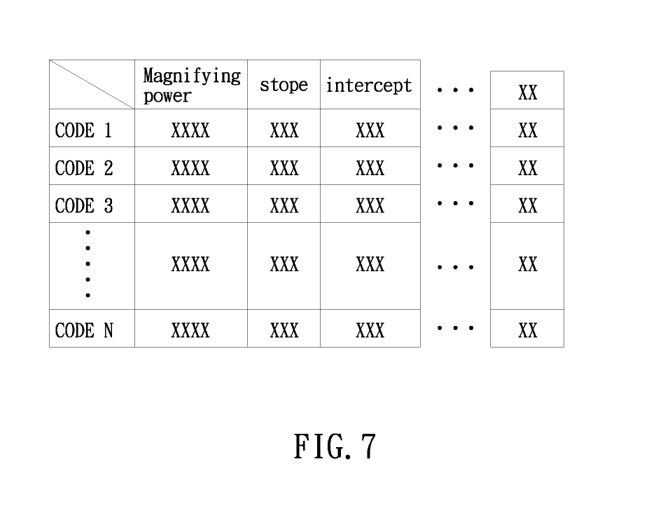

Please refer to FIG. 1, which is a schematic view of a biosensor requiring no code card according to a first embodiment of the invention. In FIG. 1, the biosensor 1, being substantially a glucose monitor, is comprised of a display unit 11, a press-key unit 12 and a test strip interface 13; wherein the display unit 11 can be a liquid crystal display (LCD) or an assembly of light emitting diodes (LEDs) that is capable of displaying a code made up of a series of characters. In a preferred embodiment, the series of characters is composed of four numerals or English alphabets, but is not limited thereby. As seen in FIG. 7, the code made up of a series of characters is corresponding to a parameter listed in the reference table stored in a parameter referencing unit of the biosensor 1, wherein the parameters stored in the parameter referencing unit includes magnifying power, slope and intercept, etc. the code is inputted into the parameter referencing unit by the pressing of the press-key unit 12. In a preferred aspect, the press-key unit 12 is composed of a top key 121, a bottom key 122 and a confirmation key 123 that can be used for configuring the glucose monitor 1 to matching a test strip with respect to its manufacturing date and thus the glucose monitor 1 is calibrated, whereas the test strip holding an enzyme therein is used for receiving blood of a patient to be test by the glucose monitor 1.

It is preferred that each character of the series of characters can be adjusted independently and individually. For instance, while the cursor of the display unit 11 is moved to the first character of the series of characters which is specified to be 0 initially, the first character can be adjusted in a circulating manner by the pressing of the top and the bottom keys 121, 122, that is, by pressing the top key 121 continuously, the first character will change in the order as following: 0→1→2 . . . →9→0; and by pressing the bottom key 122 continuously, the first character will change in the order as following: 0→9→8 . . . →1→0. As soon as a character matches the code, the confirmation key 123 is pressed for fixing the character as the first character of the series of characters and then the cursor is moved to the second character for selecting a number matching to the code, and so forth. It is noted that the abovementioned method of character adjustment is only for illustration and is not limited thereby. For instance, one may like to adjust the first two characters at the same time and then the following two characters, and so on, that is, by pressing the top key 121 continuously, the first two character will change in the order as following: 00→01→02 . . . →99→00; and by pressing the bottom key 122 continuously, the first character will change in the order as following: 00→99→98 . . . →01→00. For those skilled in the art, it is obviously that the code can contain not only numbers, but also English alphabets that are achievable by the aforesaid adjusting method.

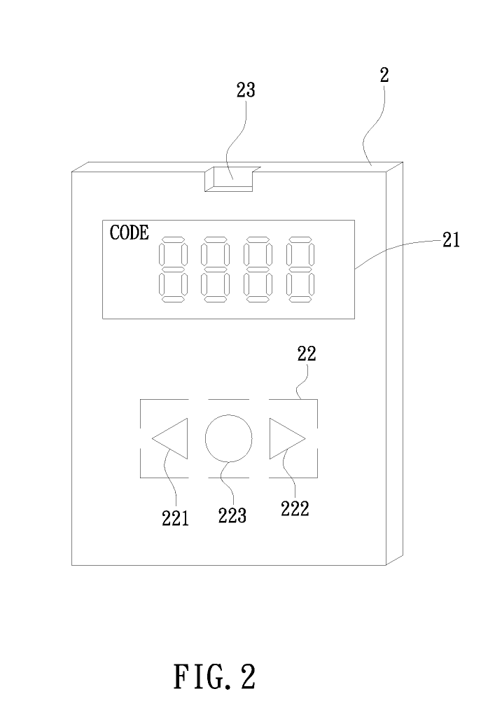

Please refer to FIG. 2, which is a schematic view of a biosensor requiring no code card according to a second embodiment of the invention. The difference between the first embodiment of FIG. 1 and the second embodiment of FIG. 2 is that the top and the bottom keys 121, 122 of the press-key unit 12 are replaced and substituted by the left and the right keys 221, 222 of the press-key unit 22. The second embodiment is preferred since it also capable of functioning the same as that of the first embodiment, but it is space-saving.

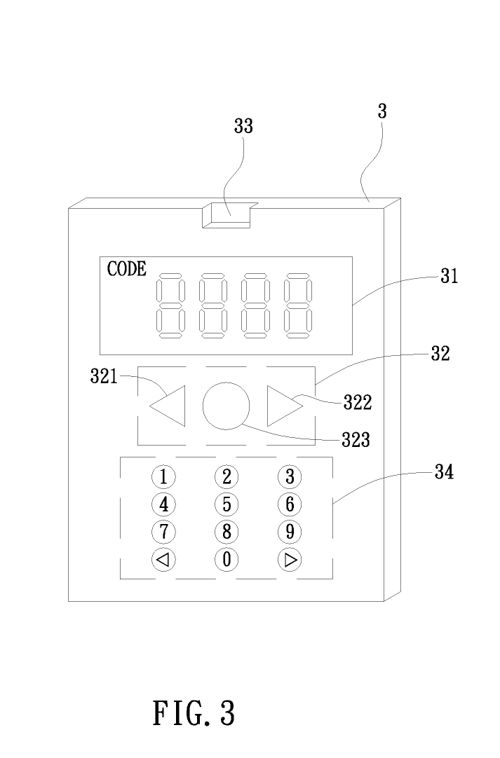

Please refer to FIG. 3, which is a schematic view of a biosensor requiring no code card according to a third embodiment of the invention. The difference between the third embodiment of FIG. 3 and the second embodiment of FIG. 2 is that there is an addition numeral keyboard 34 arranged right under the press-key unit 32, whereas the addition numeral keyboard 34 is comprised of nine number keys, respectively specified to be 0, 1, . . . , 9, and two function keys, respectively specified to be [symbol] and [symbol] . The third embodiment is preferred since it also capable of functioning the same as that of the first embodiment, but it is able to input a specific number directly and thus is more convenient.

For those skilled in the art, it is obvious that the confirmation key can be eliminated from the press-key units of FIG. 1 and FIG. 2 without affecting the input of the code made up of a specific series of characters. For instance, while the cursor of the display unit 11 is moved to the first character of the series of characters which is specified to be 0 initially and is blinking, the first character can be adjusted by the pressing of the press-key unit and as soon as the correct character is selected, no pressing is performed for a few seconds until the blinking of the first character stops that represents the first character is confirmed and then the cursor moves to the second character, and so forth.

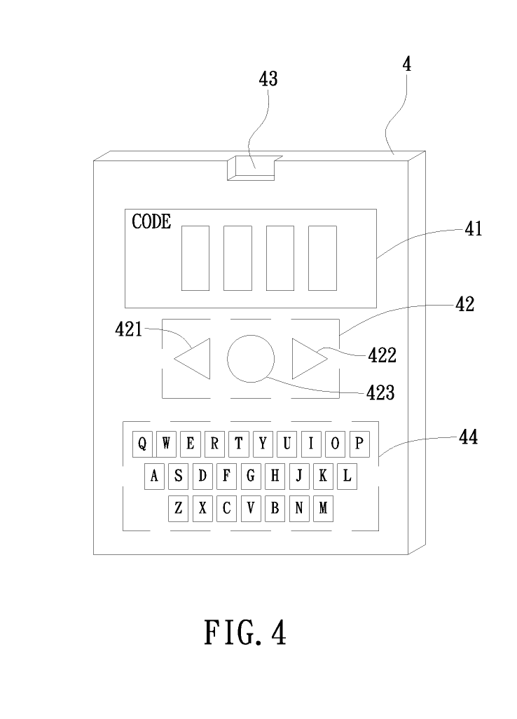

Please refer to FIG. 4, which is a schematic view of a biosensor requiring no code card according to a fourth embodiment of the invention. The difference between the fourth embodiment of FIG. 4 and the second embodiment of FIG. 2 is that there is an addition English alphabet keyboard 44 arranged right under the press-key unit 42, whereas the addition English alphabet keyboard 44 is comprised of 26 keys, respectively specified to be A to Z. The fourth embodiment is preferred since it also capable of functioning the same as that of the first embodiment, but it is able to input a specific English alphabet directly and thus is more convenient.

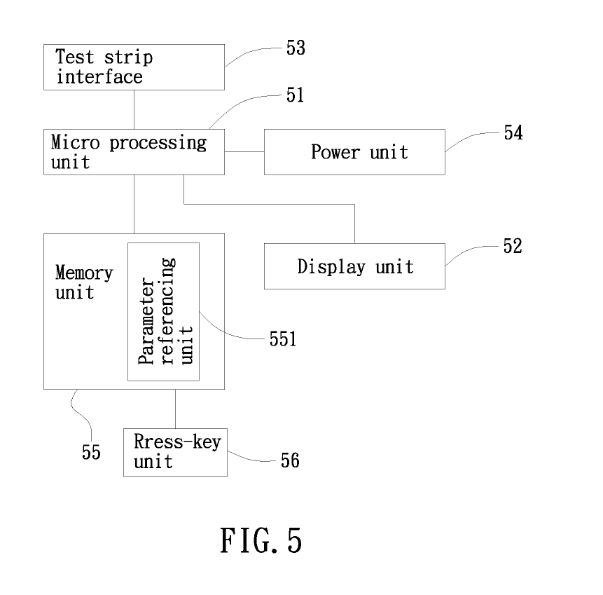

Please refer to FIG. 5, which is a first function block diagram of a biosensor requiring no code card according to the present invention. In FIG. 5, the biosensor requiring no code card is comprised of: a micro processing unit 51; a memory unit 55; a display unit 52; a power unit 54; a test strip; a test strip interface 53, for receiving the test strip; a press-key unit 56, further comprising a plurality of keys as those shown in FIG. 1 to FIG. 4, for inputting a specific series of characters, whereas each character can be a number or an English alphabet and the specific series of characters can be find on the package of a test strip, such as the casing, the rubber cover and the instructions leaflet, etc.; and a parameter referencing unit 551, for receiving and matching the inputted specific series of characters to a parameter stored therein while sending the matched parameter to the micro processing unit 51 for enabling the same to perform a calibration operation upon the biosensor. In a preferred aspect, the parameter referencing unit 551 is built in the memory unit 55 and the memory unit 55 can be a flash memory.

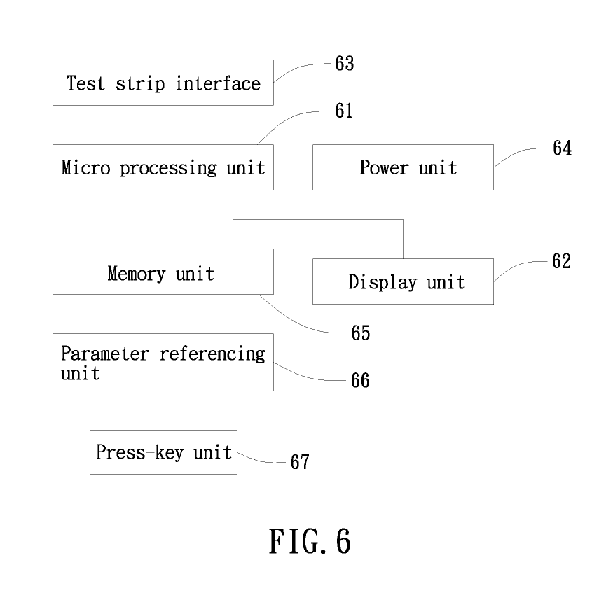

Please refer to FIG. 6, which is a second function block diagram of a biosensor requiring no code card according to the present invention. The difference between the embodiment of FIG. 6 and that of FIG. 5 is that the parameter referencing unit 66 of FIG. 6 is an independent read-only-memory (ROM) that is not built in the memory unit 65.

To sum up, as seen in FIG. 1 to FIG. 7, the present invention relates to a biosensor requiring no code card, which can be substantially a glucose monitor having a top key, a bottom key and a confirmation key arranged thereon for using the aforesaid top and bottom keys to control and select a set of English alphabet or numeral in a circulating manner and thereafter confirming and inputting a specific series of alphabets or numerals by pressing the confirmation key, whereas the specific series of alphabets or numerals is corresponding to a set of parameters stored in a parameter referencing unit arranged inside the glucose monitor and can be find on the package of a test strip. As soon as the specific series of alphabets or numerals is inputted into the glucose monitor, the set of parameter corresponding thereto is send to a micro processing unit for using the same to perform a setting and calibration operation so as to enhancing the accuracy of the glucose monitor. In a preferred embodiment, the top and the bottom keys can be replaced and substituted by a left key and a right key. In addition, the biosensor of the invention is capable of achieving multiple analyte detections in physiological fluids, e.g. blood test or urine test, for measuring glucose, urea, cholesterol, calcium, pyruvate, serum bilirubin, and so on.

While the preferred embodiment of the invention has been set forth for the purpose of disclosure, modifications of the disclosed embodiment of the invention as well as other embodiments thereof may occur to those skilled in the art. Accordingly, the appended claims are intended to cover all embodiments which do not depart from the spirit and scope of the invention.

What is claimed is:

1. A biosensor requiring no code card, comprising:
a micro processing unit;
a memory unit;
a display unit;
a power unit;
a test strip;
a test strip interface, for receiving the test strip;
a plurality of keys, for inputting a specific series of characters; and
a parameter referencing unit, for receiving and matching the inputted specific series of characters to a parameter stored therein while sending the matched parameter to the micro processing unit for enabling the same to perform a calibration operation upon the biosensor.

2. The biosensor of claim 1, wherein each character of the specific series of characters is a character selected from the group consisting of English alphabets and numerals.

3. The biosensor of claim 1, wherein the assembly of the plural keys forms a complete set of English alphabet keyboard.

4. The biosensor of claim 1, wherein the assembly of the plural keys forms a complete set of numeral keyboard.

5. The biosensor of claim 1, wherein the plural keys includes a top key, a bottom key and a confirmation key.

6. The biosensor of claim 1, wherein the plural keys includes a top key and a bottom key.

7. The biosensor of claim 1, wherein the plural keys includes a left key, a right key and a confirmation key.

8. The biosensor of claim 1, wherein the plural keys includes a left key and a right key.

9. The biosensor of claim 1, wherein the specific series of characters is specified on a package of the test strip.

10. The biosensor of claim 9, wherein the package is an item selected from a casing, a rubber cover and an instructions leaflet.

11. The biosensor of claim 1, wherein the parameter referencing unit is a read-only-memory (ROM).

12. The biosensor of claim 1, wherein the parameter referencing unit is built in the memory unit.

13. The biosensor of claim 1, wherein the parameters stored in the parameter referencing unit includes magnifying power, slope and intercept.

14. The biosensor of claim 1, being applicable to a test selected from the group of analyte detections in physiological fluids consisting of a glucose test, a urea test, a urine test, a saliva test, a perspiration test, a cholesterol test, a calcium test, a pyruvate test, a serum bilirubin test.
